(19) United States
(12) Patent Application Publication
(10) Pub. No.: US 2003/0176531 A1
(43) Pub. Date: Sep. 18, 2003

(54) Cured uncured boxfill

(76) Inventor: Dorothy Kassab, Canastota, NY (US)

Correspondence Address: Dr. Dorothy Kassab, 3503 Lincoln Knolls East, Canastota, NY, 13032, US

(21) Appl. No.: 10/096,973

(22) Filed: Mar. 14, 2002

(51) Int. Cl. A61K 6/08

(52) U.S. Cl. 523/116

(57) ABSTRACT

Cured Uncured Boxfill is a mixture of cured (solid) composite particles and uncured (viscous) composite paste. The improvement with Cured Uncured Boxfill is that it allows the composite to be condensed, especially in the interproximal box in Class II restorations, which aids in establishing a contact with the adjacent tooth. Also, it decreases overall shrinkage of the restoration because a large part of the composite is already cured/solid and has shrunk to its maximum capacity. Lastly, since any composite that already exists can be used to produce the new combination of Cured Uncured Boxfill, this material can safely be used like other composites on the market because it does not change the important, already established, properties of those composites such as wear resistance and strength, thermal expansion, hydrolytic stability.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Cured Uncured Boxfill is a novel mixture of composite material. Composite is a general term given to a restorative material used in the field of Dentistry. Composite is a 3 dimentional combination of at least 2 chemically different materials with a distinct interface separating the components. It is made up of an inorganic filler that has been added to a resin matrix. The filler is meant to upgrade the properties of the resin matrix. The geometry of the dispersed phase in terms of the shape, size, orientation, concentration, and distribution has an impact on the restoration's final cured properties such as wear resistance, thermal expansion, hydrolytic stability, viscosity, shrinkage, and hardness. Dental products manufacturers have designed the composites with optimal properties by careful selection of fillers, and ideal ratios of filler to resin matrix.

Most commonly used material for Class II restorations is amalgam. This restorative material is a suspension of alloy metals in mercury. The alloy is manually condensed by packing with a condensing instrument. As the alloy is packed, the mercury rises to the surface, which results in a more dense mass that optimally fills the matrixed cavity area. Most importantly, in condensing subsequent increments, the matrix band can be pressed against the contact point of the adjacent tooth to insure positive contact with the material after the band is removed.

There is an increased use of composite material in posterior restorations. Composite is sold in a viscous/paste form, then placed in a cavity, and finally cured to form a solid. In its viscous/paste physical state, it does not respond to condensing forces and it shrinks 1 to 3% after curing. Most frequent problem and complaint by Dentists about the composites is that it is difficult to establish a contact between adjacent teeth in Class II restorations due to

1) inability to condense composite because it is viscous/flowy (not dense enough),

2) shrinkage of 1 to 3% by volume after curng

3) alteration of the combination of filler-to-resin matrix alters wear resistance, hydrolytic stability, shrinkage, and hardness

### BRIEF SUMMARY OF THE INVENTION

The objective of this invention is the creation of a mixture of composite, Cured Uncured Boxfill, that contains two physical states: a combination of solid (cured composite particles) and viscous (uncured composite paste) of the same composite material.

The advantages provided by Cured Uncured Boxfill are as follows:

1) allow for true condensability of the material

2) decrease the overall shrinkage of the restoration after curing

3) no alteration of the composite properties such as wear resistance, expansion, and hydrolytic sensitivity

The Cured Uncured Boxfill is condensable in the following manner. The solid, cured particles are condensed in the cavity preparation, while the paste fills the voids between the solid particles, and the excess paste rises to the top. The decreased shrinkage of the final restoration occurs in the following manner. Large part of the Cured Uncured Boxfill is made up of solid particles (already cured composite) and will not shrink anymore even with subsequent curing. The minimal amount of shrinkage that will occur, when the restoration is finally cured, is from the uncured viscous/paste form of composite that is mixed with the cured/solid particles. As a result, after final curing, the overall restoration will shrink significantly less then a fully viscous/paste composite restoration after curing. The important composite properties such as wear resistance, thermal expansion, and hydrolytic activity are unchanged in the final restoration composed of Cured Uncured Boxfill composite. This is because the Cured Uncured Boxfill is made up of the same composite material but in two different physical states (cured and uncured).

### BRIEF DESCRIPTION OF THE SEVERAL VIEWS OF THE DRAWING

Not applicable

### DETAILED DESCRIPTION OF THE INVENTION

Cured Uncured Boxfill can be made in the following way. Uncured vicous/paste composite will be cut into various size and shapes, as desired by the manufacturer. Next, the pre-cut composite will be cured to form solid particles. Finally, the cured solid particles will be mixed thouroughly with uncured viscous/paste composite producing the Cured Uncured Boxfill composite mixture.

There are several improvements of this mixture. The first improvement is that this material can truly be condensable. By definition, to condense a material means to compress, concentrate, and reduce material into smaller area. Condensing eliminates the void spaces between the solid particles, fully fills the cavity area, and makes the material more dense. Condensability is very important when trying to establish contacts between the restoration and the adjacent tooth, since the Dentist must force the material and mold it to contact the adjacent tooth. A restorative material that is flowy or unresponsive to condensable forces can not be forced against the matrix band. In the Cured Uncured Boxfill composite, the cured particles will be condensed together by manual packing with a dental condensing instrument. This allows maximum expansion and optimal fill of the matrixed interproximal cavity area. As a result, after the restoration is cured, the interproximal restoration material will be as close to the adjacent tooth as possible, producing a contact.

Another improvement of Cured Uncured Boxfill is that the overall shrinkage after curing will decrease. Normally, after curing, composite shrinks between 1 to 3% by volume, depending on the composition of filler to resin matrix. Because Cured Uncured Boxfill that contains already cured composite particles (which already maximally shrunk, and will not change size even after subsequent curing of the paste that they are mixed with), the overall shrinkage of the restoration will decrease.

Last improvement of Cured Uncured Boxfill is that the properties of the finally cured composite material are not changed. This is because Cured Uncured Boxfill is made up of the same composite in two different physical states (solid and viscous/paste). Once the material is finally cured, the composite will be in one physical state-a solid.

Cured Uncured Boxfill will be used in restoration of most dental cavity preparations. It's use will be most appreciated in Class II and Class III cavity preparations, where an interproximal contact between the restoration and adjacent tooth must be established. The operative procedure will be as follows. The cavity preparation will be matrixed and wedged. Next the tooth is treated for composite bonding. Flowable composite is placed in the gingival box area and cured. Cured Uncured Boxfill is placed incrementally in the box area and in the rest of the cavity preparation. Cured Uncured Boxfill is condensed and the pressure is forced against the matrix band, especially against the contact area. The viscous/paste part of the Cured Uncured Boxfill is forced to the top where it is carved to proper occlusal anatomy. Finally the Cured Uncured Boxfill is conventionally cured with a curing light.

What distinguishes Cured Uncured Boxfill from other restorative composites is that all other composites are prepared and sold in a viscous/paste form, which is cured/solidified after final placement in the cavity preparation. There are no composite preparations, like the Cured Uncured Boxfill, that contain a mixture of solid/viscous states of composite in one.

1. What I claim as my invention is a new mixture of two physical states of composite in one, namely a suspension of a solid (cured) composite mixed with a viscous/paste (uncured) composite called Cured Uncured Boxfill.
